(19) United States
(12) Patent Application Publication
(10) Pub. No.: US 2007/0043115 A1
(43) Pub. Date: Feb. 22, 2007

(54) Use of choline to prevent thrombosis associated with total parenteral nutrition

(76) Inventor: Alan L. Buchman, Chicago, IL (US)

Correspondence Address: MCDERMOTT WILL & EMERY LLP, 600 13TH STREET, N.W., WASHINGTON, DC, 20005-3096, US

(21) Appl. No.: 11/496,710

(22) Filed: Aug. 1, 2006

Related U.S. Application Data
(60) Provisional application No. 60/708,395, filed on Aug. 16, 2005.

(51) Int. Cl. A61K 31/205; A61K 31/14

(52) U.S. Cl. 514/554; 514/642

(57) ABSTRACT

Choline deficiency is a risk factor for the development of thrombosis in patients with intestinal failure that require total parenteral nutrition. There is provided a method of preventing thrombosis, preferably venous thrombosis, comprising administering to a patient receiving TPN a nutrient solution comprising choline. Also provided is a method of diagnosing a risk for thrombosis comprising taking a sample from a patient and detecting the level of plasma-free choline.

### RELATED APPLICATION

This application claims priority to U.S. Application Ser. No. 60/708,395, filed Aug. 16, 2005, which is hereby incorporated in its entirety.

### FIELD OF THE INVENTION

The invention relates to methods of preventing thrombosis, preferably venous thrombosis. More particularly the invention relates to the use of choline to decrease homocysteine levels and prevent thrombosis in patients receiving total parenteral nutrition (TPN).

### BACKGROUND OF THE INVENTION

TPN originated as an emergency procedure which was first used following surgery for severe and massive trauma of the gastrointestinal tract. TPN has become a relatively common means of providing bowel rest and nutrition in a variety of conditions. Although TPN was initially employed as a short-term temporary nutrition procedure, it has also become widely used as a long-term nutrition protocol.

Parenteral nutrition, whether it be total or supplemental, has been employed in a wide variety of chronic conditions. For example, patients suffering malnutrition from acute and chronic inflammatory bowel diseases many times require total parenteral nutrition. In addition, patients suffering from partial or total obstruction of the gastrointestinal tract that cannot be relieved immediately by surgery are also candidates for TPN. Other patients who receive TPN include those suffering from massive burns that produce critical protein loss and those patients suffering from other disorders in which malnutrition is a threat to their life and they cannot receive or absorb nutrients via the digestive tract.

The nutrient solution or mix which is administered intravenously to the patient during TPN is generally tailored to the individual needs and tolerance of the patient. In general, the nutrient solution is an aqueous solution containing dextrose, amino acids, electrolytes, trace elements and vitamins. Two to three liters of the nutrient solution are administered intravenously to the patient during total parenteral nutrition. Administration of the nutrient solution is generally accomplished by way of a central venous catheter which is inserted in the superior or inferior vena cava.

Although total parenteral nutrition is a lifesaving feeding program for many patients, there are a number of complications which may develop. The patient may suffer adverse reactions due to sensitivity to some of the elements in the nutrient mix and the possibility of infections always exists. Other complications that may develop include choline deficiency, electrolyte imbalance, hyperglycemia, cardiac overload, dehydration, metabolic acidosis, mechanical trauma to the heart, metabolic bone disease and renal diseases.

In addition to the above noted possible complications, it has been noted that patients with intestinal failure who require long-term TPN tend to develop phlebitis and catheter thrombosis as a complication. Central venous catheter thrombosis is a frequent complication in patients who require long-term total parenteral nutrition (TPN). Buchman A L, Goodson B, Herzog F, Ament M E. Catheter thrombosis and superior/inferior vena cava syndrome are rare complications of long-term parenteral nutrition. (1994) Clinical Nutrition. 13:356-360. Recurrent thrombosis and loss of at least two major vessels is a Medicare-approved indication for small intestinal transplant. Buchman A L, Scolopio J, Fryer J. Technical review of the treatment of short bowel syndrome and intestinal transplantation. (2003) Gastroenterology 124:1111-1134. Hyperhomocysteinemia is a risk factor for development of venous thrombosis. Den Heijer M, Koster T, Blom H J, et al. Hyperhomocysteinemia as a risk factor for deep-vein thrombosis. (1996) New Engl J Med 334:759-762; Eichinger S. Homocysteine, vitamin B6 and the risk of recurrent venous thromboembolism. (2003) Pathophysiol Haemost Thromb 33:342-4; Unlu Y, Keles S, Becit N, et al. Hyperhomocysteinaemia as a risk factor for deep-vein thrombosis. Eur J Vasc Endovasc Surg 2005 (in press). Choline deficiency has previously been described in patients who require long-term parenteral nutrition and manifests in hepatic (Buchman A L, Dubin M, Jenden D, et al. Lecithin supplementation causes a decrease in hepatic steatosis in patients receiving long term parenteral nutrition. (1992) Gastroenterology 102:1363-70; Buchman A L, Dubin M, Moukarzel A, et al. Choline deficiency: a cause of hepatic steatosis associated with parenteral nutrition that can be reversed with and intravenous choline chloride supplementation. (1995) Hepatology 22:1399-1403; Buchman A L, Sohel M, Dubin M, Jenden D J, Roch M. Choline deficiency causes reversible hepatic abnormalities in patients during parenteral nutrition: proof of a human choline requirement; a placebo-controlled trial. (2001) JPEN 25:260-268; Buchman A L, Sohel M, Brown M, et al. Verbal and visual memory improve after choline supplementation in long-term total parenteral nutrition: a pilot study. (2001) JPEN 25:30-35) and neuropsycholic abnormalities. Buchman A L, et al. (2001) JPEN 25:30-35. In rodents, choline deficiency is associated with hyperhomocysteinemia. Da Costa K A, Gaffney C E, Fischer L M, Zeisel S H. Choline deficiency in mice and humans is associated with increased plasma homocysteine concentration after a methionine load. (2005) Am J Clin Nutr 81:440-444; Chen A H, Innis S M, Davidson A G, James S J. Phosphatidycholine and lysophosphatidylcholine excretion is increased in children with cystic fibrosis and is associated with increased plasma homocysteine, S-adenosylhomocysteine, and S-adenosylmethionine. (2005) Am J Clin Nutr 81:686-691; Monserrat A J, Musso A M, Tartas N, et al. Consumption coagulopathy in acute renal failure induced by hypolipotropic diets. (1981) Nephron 28:276-284; and Varela-Moreiras G, Ragel C, Perez de Miguelsanz J. Choline deficiency and methotrexate treatment induces marked but reversible changes in hepatic folate concentrations, serum homocysteine and DNA methylation rates in rats. (1995) J Am Coll Nutr 14:480-485. Oral choline supplementation decreased plasma homocysteine concentration in a group of patients with premature arterial vascular disease (Dudman N P, Wilcken D E, Wang J, et al. Arterial thrombosis and venous thrombosis are different events however, arterial thrombosis being quite rare and isolated, whereas venous thrombosis is relatively comment in patients receiving TPN. Disordered methionine/homocysteine metabolism in premature vascular disease. Its occurrence, cofactor therapy, and enzymology. (1993) Arterioscler Thromb 13:1253-1260.) and in a group of healthy men (Olthof M R, Brisk E J, Katan M B, Verhoef P. Choline supplemented as phosphatidylcholine decreases fasting and postmethionine-loading plasma homocysteine concentrations in healthy men. (2005) Am J Clin Nutr 82:111-117.). Therefore, choline deficiency is a suspected significant risk factor for development of catheter thrombosis in patients with intestinal failure risk factor in the development of catheter thrombosis.

It is, therefore, desirable to provide a simple and effective treatment which is capable of increasing plasma-free choline levels in patients receiving total parenteral nutrition to decrease homocysteine levels in the blood. Further, it is desirable to provide a treatment which is effective in reducing hyperhomocysteinemia, thereby preventing catheter thromboses which are associated with choline deficiency resulting from long-term total parenteral nutrition.

### SUMMARY OF THE INVENTION

In accordance with the present invention, it was discovered that there is an association with plasma-free choline levels in patients who receive TPN and (1) hyperhomocysteinemia, and (2) incidence of catheter thrombosis. We show that a reduced or below normal level of plasma-free choline levels in patients receiving total parenteral nutrition correlates with a risk or increased risk for catheter thrombosis, primary and recurrent thrombosis. Others have shown the risk of venous thrombosis with elevated homocysteine levels. It is therefore reasoned that reduced or below levels of plasma-free choline is associated with hyperhomocysteinemia. Plasma-free choline levels increased to or near normal levels and/or maintained at or near normal levels by e.g., including choline in the nutrient solution which is administered parenterally to the patient would therefore, not only maintain plasma-free choline levels in a normal range, but would also be effective in reducing homocysteine levels in the blood, and thereby prevent and/or inhibit catheter thrombosis.

In one aspect of the present invention, choline, preferably in the form of choline chloride or other choline salt, is administered to the patient receiving total parenteral nutrition. The choline may be added to the nutrient solution or be separately infused parenterally. The normal dosage of nutrient solution administered to the patient on a daily basis is on the order of two to three liters of solution. As a feature of the present invention, a therapeutically effective amount of choline chloride or other choline salt is infused parenterally or added to a nutrient solution in an amount sufficient to provide from 0.25 to about 8 grams of choline per liter of solution.

The above concentrations of choline in the nutrient solution are tolerated well by patients and provide a daily dosage of choline which is effective in maintaining plasma-free choline levels within normal ranges during chronic total protein nutrition therapy. U.S. Pat. No. 5,567,736. It was discovered that addition of choline to the nutrient solution at the preceding dosage levels is effective in reducing homocysteine levels and preventing or inhibiting catheter thrombosis in those patients suffering from chronic choline deficiency.

As a feature of the present invention, an improved nutrient solution is provided by adding sufficient choline chloride or other salt of choline to the TPN patient's normal nutrient solution. No complicated formulation procedures or difficult mixing steps are necessary to carry out the present invention. The desired dosage of choline chloride or other salt of choline is added to the aqueous nutrient solution with the choline enriched solution being administered to the TPN patient in accordance with normal practice.

Choline chloride does not react with or otherwise adversely affect the dextrose, amino acids, electrolytes, trace elements, vitamins and other compounds typically found in total parenteral nutrient solutions. In addition, choline is relatively stable within the nutrient solution when stored under normal conditions. The choline does not deteriorate or otherwise lose its potency over relatively long periods of time. As a result, choline may be added to the TPN nutrient solution by a pharmacist or physician, or it can be added by the patient immediately prior to administration of the solution. The use of choline as a supplement to TPN nutrient solutions is easily incorporated into situations requiring both long-term and short-term total parenteral nutrition.

In another aspect of the invention there is provided kits for reducing homocysteine levels and/or preventing thrombosis, preferably venous thrombosis, in a patient receiving TPN comprising a nutrient solution and a therapeutically effective amount of choline generally comprised in a container.

In another aspect of the invention there is a method of monitoring choline levels in a patient receiving TPN to diagnose a risk of thrombosis, preferably venous thrombosis. In a preferred embodiment the method comprises obtaining a sample of blood or plasma from a patient receiving TPN and detecting the level of choline, wherein a low or decreased level of plasma-free choline in comparison to normal levels is indicative of an increased risk of thrombosis. Choline levels may be monitored over time to detect a trend in choline levels as a determinative factor in risk of thrombosis, or to monitor effectiveness of choline infusion as a prophylaxis to decrease risk of thrombosis.

The above-discussed and many other features and attendant advantages of the present invention will become better understood by reference to the detailed description when taken in conjunction with the accompanying drawings.

### DETAILED DESCRIPTION OF THE INVENTION

The present invention shows for the first time that choline deficiency is associated with development of catheter thrombosis in patients with intestinal failure. In addition, a trend towards lower plasma-free choline concentration in patients with recurrent thrombosis was also observed. All publications, patents and patent applications cited herein, whether supra or infra, are hereby incorporated by reference in their entirety.

For the purposes of this specification and the appended claims, the term “choline” includes choline, choline salts, choline precursors and choline metabolites, wherein the choline precursors or choline metabolites are capable of being converted into choline when mixed with a TPN solution or introduced in vivo.

As used herein and in the appended claims, the term “normal level” means choline within the range of about 10 nmol/ml to about 15 nmol/ml.

As used herein and in the appended claims, the term “parenteral” and “parenterally” means by some other means than through the gastrointestinal tract; referring particularly to the introduction of substances into a patient by intravenous, subcutaneous, intramuscular, or intramedullary injection.

As used herein and in the appended claims, the term “patient” includes members of the animal kingdom including but not limited to human beings.

As used herein and in the appended claims, the term “therapeutically effective amount” means an amount sufficient to increase plasma-free choline to or near a normal level.

As used herein and in the appended claims “thrombosis” means the formation or presences of clotting within a blood vessel which may cause infarction of tissues supplied by the vessel. Preferably, thrombosis refers to venous thrombosis of the superior and/or inferior vena cava. Most preferably, thrombosis refers to venous catheter thrombosis, primary and/or recurrent.

In one aspect of the present invention, there is provided a method of decreasing homocysteine levels in a patient receiving total parenteral nutrition (TPN) comprising administering parenterally to the patient a therapeutically effective amount of choline. In another aspect of the invention there is provided a method of preventing thrombosis, preferably venous thrombosis, in a patient receiving total parenteral nutrition (TPN) comprising administering parenterally to the patient a therapeutically effective amount of choline. Preferably, the therapeutically effective amount of choline decreases plasma homocysteine levels in the patient. In another preferred embodiment, the therapeutically effective amount of choline prevents thrombosis.

The choline may be administered together with or separately from a nutrient solution. In one embodiment, the choline may be infused into a patient separately from a nutrient solution. Preferably, choline is administered in a total daily dosage of about 0.25 to about 8 grams. In another embodiment, a nutrient solution may be supplemented with choline, and the choline supplemented nutrient solution may be infused in to a patient.

The nutrient solutions which are used in feeding patients by total parenteral nutrition (TPN) are well known. The particular ingredients which are included in the nutrient solution vary widely depending upon patient nutritional needs and the patient's medical condition. Generally, the most prevalent ingredient in nutrient solutions is dextrose with lesser amounts of amino acids, electrolytes, trace elements and vitamins being included. In general, the nutrient solution will include 10-35 volume percent of a dextrose solution. Dextrose solutions are aqueous-based solutions which contain between 10 and 35 grams of dextrose per liter of solution. Such dextrose solutions are commercially available from Abbott Laboratories, Baxter Healthcare Corp., McGaw Laboratories and others.

Nutrient solutions also typically include from about 2-5 weight percent of amino acids. The amino acids used in the nutrient solution can be any of the essential amino acids and can be included in a variety of concentrations and mixtures. Preferred amino acids for use in nutrient solutions include threonine, serine, proline, glycine, alanine, valine, methionine, isoleucine, leucine, tyrosine, phenylalanine, tryptophan, lysine, histidine and arginine. In addition to dextrose and amino acids, most nutrient solutions include electrolytes such as potassium, sodium, chloride, magnesium, acetate, calcium, and phosphorous and others. These electrolytes are generally included in relatively small amounts on the order of 1 percent or less.

Trace elements and vitamins are also included in most nutrient solutions. Typical trace elements include selenium, chromium, manganese, zinc and others. Vitamins which are usually included in nutrient solutions include A, B₁, B₂, B₆, B₁₂, C, D, E, biotin, pantothenic acid, etc. Trace elements are usually present in concentrations on the order of less than one-tenth of one percent. Vitamins are generally present in amounts sufficient to meet daily vitamin requirements which have been established and are widely known.

In accordance with the present invention, choline, preferably choline chloride or another choline salt, is added to the nutrient solution to provide for direct intravenous introduction of choline into the patient. Although choline chloride is the preferred form of choline which is added to the nutrient solution, other choline salts such as choline bitartrate, choline dihydrogen citrate, choline phosphate and choline bicarbonate, as well as, other pharmaceutical salts understood by those skilled in the art, may be used. In addition, choline precursors and choline metabolites such as phosphatidyl choline, CDP-choline, soy lecithin, etc., may be used.

The amount of choline which is added to the nutrient solution may be varied depending upon the patient's plasma-free choline level, the degree of hyperhomocysteinemia and the severity of other medical problems associated with choline deficiency.

In general, it is preferred that the amount of choline which is added to the nutrient solution be sufficient to provide a choline concentration in the solution of from about 0.25 to about 8 grams choline per liter of solution. This concentration of choline provides an acceptable daily dosage level of choline when the patient receives from 2 to 3 liters of the nutrient solution each day. The desired total daily choline dosage should be on the order of between about 0.5 to about 8 grams of choline.

Administration of the nutrient solution parenterally into the patient is accomplished by well established techniques for administration of nutrient solutions for total parenteral nutrition. The total amount of nutrient solution may be administered as separate aliqouts at different times during the day. However, it is preferred that the two to three liter dose of nutrient solution be administered each evening over a six to ten hour period, or on a continuous 24 hour basis.

The choline which is added to the nutrient solution is preferably added as a solution. The appropriate amount of choline is weighed and dissolved in sterile water for injection United States Pharmacopeia (USP) to form an approximately 50% by weight solution of choline. The resulting choline solution is then passed through a 0.2 μm pore size sterilizing filter and packaged in sterile vials. Suitable filters for filtering the choline solution include 0.2 μm Nylon 66 sterilizing filter cartridges such as those manufactured by Pall Ultrafine Corp. (Glen Cove, N.Y.). Other Nylon filters may be used as well as filters made from other materials, such as cellulose acetate and polysulfone.

Prior to use, the appropriate amount of choline solution is transferred from the sterile vial and is added to the nutrient solution which typically is contained in a TPN bag. Choline chloride has been found to be stable in various TPN nutrient solutions for at least 30 days and there were no adverse affects on the TPN solution turbidity, pH, or amino acid concentrations.

It is preferred that the aqueous solution of choline be stored separate from the TPN solution and added to the nutrient solution on the day the solution is to be prepared. The concentration of choline within the nutrient solution may be varied depending upon the total amount of solution being administered. Preferably, the amount of choline solution added to the nutrient solution administered to the patient should provide a total daily dosage of choline of between about 0.5 to 8 grams of choline. Total daily dosages on the order of 1-6 grams of choline will be adequate for most treatment regimens.

The present invention also encompasses other methods of adding choline to a nutrient solution which are used or developed by one skilled in the art. Nutrient solution for TPN may also be manufactured to already include the choline as an element of its content.

In still another aspect, the present invention provides kits for preventing thrombosis in a patient who receives TPN. In one embodiment a nutrient solution and a therapeutically effective amount of choline are provided in the kit, generally comprised within a suitable container. The components of the kits may be packaged either in aqueous media. The container means of the kits will generally include at least one container means, such as a vial, test tube, flask, bag, bottle, syringe or other container means, into which the nutrient solution and the choline may be placed. Preferably the nutrient solution is maintained separate from the choline.

In yet another aspect of the present invention, there is provided methods of diagnosing a risk of thrombosis, preferably venous thrombosis. In a preferred embodiment, the method comprises taking a biological sample from a patient receiving TPN, detecting the level of choline in the sample and correlating the level of choline in the sample to the presence or absence of risk for thrombosis. Below normal levels of choline correlates with a risk of thrombosis, and the lower the choline levels, the greater the risk. Conversely, Normal or above normal levels of choline correlate to a risk free and/or low risk of thrombosis. The biological sample may include any biological fluid sample such as blood, plasma, serum and urine.

Preferably, the choline detected is plasma-free choline, however, phospholipid-bound choline and phosphorocholine may also be detected. Plasma-free choline is preferably detected using gas chromatography/mass spectroscopy (GC/MS) according to the methods described in Jenden D J, Roch M, Booth R A. Simultaneous measurement of endogenous and deuterium-labeled tracer variants of choline and acetylcholine in subpicomole quantities by gas chromatography-mass spectrometry. (1973) Anal Biochem 55:438-448, 1973 and Freeman J J, Choi R L, Jenden D J. (1975) Plasma choline, its turnover and exchange with brain choline. J Neurochem 24:729-734. Radioimmunoassay (RIA) and high performance liquid chromatography (HPLC) may also be used to detect choline.

In yet another aspect of the invention there is provided a thrombosis risk monitoring method utilizing choline detection methods. Thus, for example, by measuring choline levels in biological samples from a patient receiving TPN over time, it is possible to monitor a change in risk of thrombosis and/or determine whether a particular therapeutic regimen, such as choline infusion aimed at preventing thrombosis is effective. Below normal levels of choline correlates with a risk of thrombosis, and the lower the choline levels, the greater the risk. Conversely, Normal or above normal levels of choline correlate to a risk free and/or low risk of thrombosis.

The following examples are presented for the illustrative purposes and it is to be understood that the present invention is not limited to those precise embodiments. It should be appreciated by those of skill in the art that the techniques disclosed in the examples which follow represent techniques to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. Examples of practice are as follows:

### EXAMPLE 1

### Correlation Between Hyperhomocysteinemia and Choline Deficiency

Data obtained during a prospectively-designed study of plasma-free choline concentrations in a random group of long-term home TPN patients (Buchman A L, Moukarzel A, Jenden D J, et al. Low plasma fee choline is prevalent in patients receiving long term parenteral nutrition and is associated with hepatic aminotransaminase abnormalities. (1993) Clin Nutr. 12:33-37.) and a retrospective study of the incidence of catheter thrombosis in a group of 527 home TPN patients (Buchman A L, et al., (1994) Clinical Nutrition. 13:356-360.) shows a correlation between hyperhomocysteinemia and choline deficiency. The patients studied represent a subset of those subjects, namely those that had their plasma-free choline concentrations determined in 1991. Patient diagnoses are listed below in table 1.

TABLE 1
TPN Indications

| | |
|---|---|
| Short bowel syndrome | 22 |
| Crohn's disease | 9 |
| Radiation enteritis | 8 |
| Chronic intestinal pseudoobstruction | 5 |
| Gastroschisis | 3 |
| Collagenous sprue | 3 |
| Unclassified | 3 |
| Multiple resections | 2 |
| Necrotizing enterocolitis | 2 |
| Retroperitoneal sarcoma | 1 |
| Mesenteric venous thrombosis | 1 |
| Mesenteric arterial thrombosis | 1 |
| Exomphalos | 1 |
| Midgut volvulus | 1 |
| Dumping syndrome | 1 |
| Chylous ascites/abdominal trauma | 1 |
| Colonic interposition for esophageal carcinoma | 1 |

All catheters used in the subject patients were Hickman® or Broviac®-type; there were no percutaneous-inserted central catheters (PICCs). Most, but not all were single-lumen, although the exact number of single lumen catheters used was indeterminable because of inadequate documentation. Catheter tip was verified in either the superior or inferior vena cava by fluoroscopy at the time of insertion, and subsequently in most, but not all patients by chest radiograph. TPN consisted of a single 1-3 L bag containing a mixture of dextrose (15-25% final concentration), free amino acids (3.5-5% final concentration), electrolytes, minerals, trace metals, and multivitamins. Lipid emulsion (20%) was delivered in a “piggybacked” style; no 3:1 emulsions were used. All patients except one received cyclic nighttime infusion over 8-12 hours 3-7 days per week depending on individual nutrient and/or fluid requirements. Heparin was routinely used as a catheter flush in all patients. No patient was receiving heparin or warfarin prior to their catheter thrombosis. Hydrocortisone was not added to the TPN solution.

Catheter thrombosis that was suspected clinically on the basis of difficulty flushing or infusing the catheter, or extremity, neck, or facial edema, was confirmed with contrast venography both through the catheter and proximal to the catheter. Patients with no evidence of thrombosis with contrast venography were not included in this study. Patients with thrombosis related to a malpositioned catheter were excluded as were those with symptoms solely on the basis of localized thrombosis that resolved following instillation of urokinase 5,000 per catheter lumen were not included in the study.

41 subjects with 231 catheter years (one patient with a catheter for one year=one catheter year) were included in the study. This included 21 males (aged 34±26 yrs) that had received home TPN for 5.8±5.0 yrs and 20 females (aged 55.1±18.1 yrs) that had received home TPN for 5.6±4.1 yrs were studied. 16 (39%) patients developed catheter thrombosis and 5 had recurrent catheter thrombosis. This compared with 11% of 527 patients (1154 catheter years) in the original thrombosis study. (Buchman A L, et al., (1994) Clinical Nutrition. 13:356-360.) Age, sex, TPN duration, and TPN indication were similar between patients who developed catheter thrombosis and those that did not.

A thrombophilia evaluation was undertaken with measurement of platelet count, protein C, protein S, plasminogen, antithrombin III concentrations, and anticardiolipin and antiphospholipid antibodies using standard techniques. Thrombocytosis was defined as a platelet count of >450,000/mL.

Heparinized blood was obtained for plasma free and phospholipid-bound choline determination following an overnight 8-10 hour fast. Specimens were placed immediately on ice and centrifuged at 3000×g at 4° C. within 20 minutes of collection; plasma was decanted off. Plasma-free choline was determined using gas chromatography/mass spectroscopy (GC/MS) (Jenden D J, Roch M, Booth R A. Simultaneous measurement of endogenous and deuterium-labeled tracer variants of choline and acetylcholine in subpicomole quantities by gas chromatography-mass spectrometry. (1973) Anal Biochem 55:438-448; and Freeman J J, Choi R L, Jenden D J. Plasma choline, its turnover and exchange with brain choline. (1975) J Neurochem 24:729-734.), and phospholipid-bound choline was determined following extraction as described by Folch et al. (A simple method for the isolation and purification of lipids from animal tissue. (1957) J Biol Chem 226:497-509.), and hydrolysis by Jope and Jenden (Jope R S, Jenden D J. Choline and phospholipid metabolism and the synthesis of acetylcholine in rat brain. (1979) J Neurosci Res 4:69-82).

All results are presented as mean±standard deviation. A p value of <0.05 was defined as an indication of statistical significance.

Plasma-free choline was below normal (11.4±3.7 nmol/ml) in 33 of the 41 subjects, and was 7.7±2.7 nmol/ml in patients with no history of catheter thrombosis and 6.2±1.7 nmol/ml in patients with previous catheter thrombosis (p=0.076 by Wilcoxin rank-sum test). The plasma-free choline concentration tended to be lower in patients with >1 thrombosis and than in those with only a single event (6.0±1.8 nmol/ml vs 7.7±2.7 nmol/ml), although the difference was not statistically significant. The partial correlation between plasma-free choline concentration and the frequency of clots after controlling for catheter duration was r=−0.33, p=0.038.

Plasma phospholipid-bound choline concentration was normal in 34 of 41 subjects and was 2191.7±679.0 nmol/ml in patients with previous catheter thrombosis and 2103.3±531.2 nmol/ml in patients without history of catheter thrombosis (p=NS).

No patient had thrombocytosis, protein C, protein S, plasminogen, or antithrombin III deficiency, thrombocytosis, anticardiolipin or antiphospholipid antibodies, active inflammatory bowel disease or active malignancy. Factor V Leiden and prothrombin gene mutation assays were not available at the time of data collection. Homocysteine concentrations were not measured as it was unknown at that time that elevated plasma homocysteine concentration represented a risk factor for either arterial or venous thrombosis at the time the study was undertaken and many of the patients are now deceased.

All patients with catheter thrombosis were anticoagulated initially with heparin, and subsequently with warfarin following catheter removal and replacement. Not all patients with recurrent catheter thrombosis however had therapeutic prothrombin at the time of recurrence. Given that an attempt was made at anticoagulation following the initial thrombosis, it is likely this anticoagulation therapy lead to a decreased risk of subsequent thrombus formation, and therefore decreased the correlation between choline and thrombosis as choline status would remain unchanged in the face of anticoagulation. We suspect the reason for the much greater incidence of catheter thrombosis in this subset of patients from our original report (Buchman A L, et al. (1994) Clinical Nutrition. 13:356-360.), is primarily related to the fact the patients in our study had a much greater duration of TPN (5.7 yrs vs 0.6 yrs).

Although plasma homocysteine concentration was not measured in our patients, given the correlation between elevated plasma homocysteine and incidence of catheter thrombosis in TPN-dependent short bowel patients described by Compher et al. (Hyperhomocysteinemia is associated with venous thrombosis in patients with short bowel syndrome. (2001) JPEN 25:1-7.), it is likely the elevation in plasma homocysteine these investigators observed was in fact, caused by choline deficiency. Choline deficiency was likely the ultimate cause for many of the thrombotic events we observed given that our patients already received sufficient intravenous folate, pyridoxine (B6), and B12 supplementation.

Because homocysteine metabolism requires a methyl donor for re-methylation, a deficiency in methyl donors such as choline may result in hyperhomocysteinemia. The increase in homocysteine concentration observed by Compher et al. is consistent with a defect in the hepatic transulfuration pathway. That occurs when nutrients that require metabolism bypass the portal circulation and are intravenously delivered. Stegink L D, Den Besten L. Synthesis of cysteine from methionine in normal adult subjects: effect of route of administration. (1972) Science 178:514-515; and Chawla R K, Berry C J, Kutner M H, Rudman D. Plasma concentrations of transsulfuration pathway products during nasoenteral and intravenous hyperalimentaion of malnourished patients. (1985) Am J Clin Nutr 42:577-584. Although, the mechanism by which elevated plasma homocysteine concentration increases the risk for venous thrombosis is uncertain, it may involve inhibition of thrombomodulin-dependent protein C activation (Rodgers G M, Conn M T. Homocysteine, an atherogenic stimulus, reduces protein C activation by arterial and venous endothelial cells. (1990) Blood 75:895-901.) or inhibition of endothelial nitric oxide production. Stamler J S, Osborne J A, Jaraki O, et al. Adverse vascular effects of homocysteine are modulated by endothelium-derived relaxing factor and related oxides of nitrogen. (1993) J Clin Invest 91:308-318; and Upchurch G R, Walch G N, Fabian A J, et al. Homocyst(e)ine decreases bioavailable nitric oxide by a mechanism involving glutathione peroxidase. (1997) J Biol Chem 272:17012-17017.

Historically, it has been suggested that folate, vitamin B₁₂, or vitamin B₆ deficiencies alone resulted in elevated plasma homocysteine concentration. Stipanuk M H. Metabolism of sulfur-containing amino acids. (1986) Anu Rev Nutr 6:179-209; and Selhub J, Jacques P F, Wilson P W F, et al. Vitamin status and intake as primary determinants of homocysteinemia in an elderly population. (1993) JAMA 270:2693-2698. Although serum folate or vitamin B₁₂ concentrations were not measured, all of our patients received these in TPN (folic acid 0.6 mg/day, pyridoxin 6 mg, and B₁₂ 5 mg per bag). Given that these administered doses are 100% bioavailable because of the nature of their delivery as compared to much lower availability from similar oral doses, it is unlikely deficiencies of either vitamin existed in the patients.

### EXAMPLE 2

### Method of Preventing Catheter Thrombosis

A patient known to have low plasma-free choline levels and receiving home TPN for an extended time is administered a choline supplemented TPN solution. A choline salt supplemented nutrient solution, having about 0.25 to about 8 grams of choline salt per liter of solution, is administered to the patient intravenously. The amount of choline which is added to the nutrient solution may be varied depending upon the patient's plasma-free choline level, the degree of hyperhomocysteinemia and the severity of other medical problems associated with choline deficiency. The choline supplemented nutrient solution is administered as one dose over a period of 10-24 hours.

### EXAMPLE 3

### Method of Preventing Catheter Thrombosis

A patient known to have low plasma-free choline levels and receiving home TPN for an extended time is administered a therapeutically effective amount of choline. A choline salt solution, having about 0.25 to about 8 grams of choline salt per liter of solution, is administered to the patient intravenously. The amount of choline in solution may be varied depending upon the patient's plasma-free choline level, the degree of hyperhomocysteinemia and the severity of other medical problems associated with choline deficiency. The choline solution is administered as one dose over a period of 10-24 hours.

### EXAMPLE 4

### Method of Diagnosing a Risk of Catheter Thrombosis

Blood or serum samples are obtained from a patient receiving TPN and the level of plasma-free choline in the sample is detected using gas chromatography/mass spectroscopy (GC/MS) according to the methods described in Jenden D J, Roch M, Booth R A. Simultaneous measurement of endogenous and deuterium-labeled tracer variants of choline and acetylcholine in subpicomole quantities by gas chromatography-mass spectrometry. (1973) Anal Biochem 55:438-448, 1973 and Freeman J J, Choi R L, Jenden D J. (1975) Plasma choline, its turnover and exchange with brain choline. J Neurochem 24:729-734. Low or decreased level of plasma-free choline in comparison to normal levels is indicative of an increased risk of catheter thrombosis. The change in choline levels over time may also be monitored to track the effectiveness of prophylactic treatment by choline infusion and/or to detect a trend in choline levels as a determinative factor in risk of catheter thrombosis.

### EXAMPLE 5

### Method of Reducing or Preventing Hyperhomocysteinemia

Two female normal volunteers were infused with C-13-labeled choline and plasma levels of homocysteine were determined before and after infusion. Plasma concentrations of homocysteine decreased from 6.62 umol/L and 7.47 umol/L to 5.14 umol/L and 6.64 umol/L, respectively, demonstrating that administration of choline can reduce the risk of or prevent hyperhomocystenemia.

What is claimed is:

1. A method of decreasing the risk of venous thrombosis in a patient receiving total parenteral nutrition (TPN) comprising parenterally administering to the patient receiving TPN a therapeutically effective amount a nutrient solution comprising choline.

2. The method according to claim 1 wherein the therapeutically effective amount of choline decreases plasma homocysteine levels in the patient.

3. The method according to claim 1 wherein the nutrient solution comprises about 0.25 to about 8 grams of choline salt per liter of solution.

4. The method according to claim 1 wherein the choline in the nutrient solution is a pharmaceutically acceptable salt of choline, choline precursor, choline metabolite or a combination thereof.

5. The method according to claim 1 wherein the choline in the nutrient solution is a choline salt.

6. The method according to claim 5 wherein the choline salt is choline chloride, choline bitartrate, choline dihydrogen citrate, choline phosphate or choline bicarbonate.

7. A method of preventing catheter thrombosis associated with total parenteral nutrition (TPN) comprising parenterally administering to a patient receiving TPN a therapeutically effective amount of a nutrient solution comprising choline.

8. The method according to claim 7 wherein the therapeutically effective amount of choline decreases plasma homocysteine levels in the patient.

9. The method according to claim 7 wherein the nutrient solution comprises about 0.25 to about 8 grams of choline salt per liter of solution.

10. The method according to claim 7 wherein the choline in the nutrient solution is a pharmaceutically acceptable salt of choline, choline precursor, choline metabolite or a combination thereof.

11. The method according to claim 10 wherein the choline in the nutrient solution is a choline salt.

12. The method according to claim 11 wherein the choline salt is choline chloride, choline bitartrate, choline dihydrogen citrate, choline phosphate or choline bicarbonate.

13. A method of determining whether a patient receiving TPN is at risk for catheter thrombosis comprising taking a sample of blood or plasma from the patient, detecting the level of plasma-free choline in the sample, and correlating the level of plasma-free choline in the sample to the presence or absence of risk for thrombosis.

14. The method according to claim 13 wherein the level of plasma-free choline is detected using gas chromatography and mass spectroscopy, RIA or HPLC.

15. The method according to claim 13 wherein a reduced level of choline in comparison to normal levels is indicative of a risk for catheter thrombosis.

16. A kit for preventing catheter thrombosis in a patient receiving TPN comprising, a nutrient solution and a therapeutically effective amount of choline.

17. An aqueous nutrient solution comprising dextrose, electrolyte, amino acids, trace elements, vitamins and a therapeutically effective amount of choline.

18. The nutrient solution of claim 17 wherein the choline is a choline salt.

19. The nutrient solution of claim 18 wherein the choline salt is choline chloride, choline bitartrate, choline dihydrogen citrate, choline phosphate or choline bicarbonate.

20. The nutrient solution of claim 17 wherein the choline is present in an amount of from about 0.25 to about 8 grams per liter of the nutrient solution.
